(19) United States
(12) Patent Application Publication
(10) Pub. No.: US 2026/0283263 A1
(43) Pub. Date: Sep. 24, 2026

(54) FRESH AIR DEVICE AND FRESH AIR DEVICE ASSEMBLY

(71) Applicant: Shenzhen Zheyang Technology Co., Ltd., Shenzhen (CN)

(72) Inventor: Min Chen, Shenzhen (CN)

(73) Assignee: Shenzhen Zheyang Technology Co., Ltd., Shenzhen (CN)

(21) Appl. No.: 19/214,515

(22) Filed: May 21, 2025

(30) Foreign Application Priority Data
Mar. 24, 2025 (CN) 202510352000.6

(51) Int. Cl. A24F 47/00; F16M 11/04; H01F 7/02

(52) U.S. Cl. CPC A24F 47/00; F16M 11/04; H01F 7/0252

(57) ABSTRACT

The present invention relates to a fresh air product, in particular to a fresh air device and a fresh air device assembly. The fresh air device includes a casing and a cartridge arranged in the casing, and further includes: an air inlet valve, arranged at one end of the casing; an air outlet valve detachably arranged at the other end of the casing; and a mouthpiece arranged on the air outlet valve. According to the fresh air device provided by the present invention, the air inlet valve and the air outlet valve are arranged at two ends to seal the casing and control the overall gas quantity, such that liquid in the cartridge may be effectively prevented from being volatilized and escaping, thus prolonging the service life of the cartridge; moreover, the overall gas quantity may be easily controlled as required, and the fresh air device is reusable and more environmentally friendly.

### CROSS-REFERENCE TO RELATED APPLICATIONS

The application claims priority of Chinese patent application CN202510352000.6, filed on Mar. 24, 2025, which is incorporated herein by reference in its entireties.

### BACKGROUND OF THE INVENTION

### 1. Technical Field

The present invention relates to a fresh air product, in particular to a fresh air device and a fresh air device assembly.

### 2. Description of Related Art

Fresh air devices are mainly used for throat nourishing, refreshing and oral care and are suitable for long-term use particularly for people who are not comfortable with their pharynx and throat or who need to freshen their breath. The main component of raw liquor used for the fresh air devices is herbal extract or fruity liquid and traditional Chinese medicine liquid. The fresh air devices may also be used for smoking cessation.

An existing fresh air device typically includes a mouthpiece, a casing and a cartridge. The mouthpiece is detachably connected to the casing. The cartridge is arranged in the casing. Raw liquor is adsorbed on the cartridge. Through-holes are formed in two ends of the casing. Air flows in from the end, away from the mouthpiece, of the casing and flows out from the end, connected to the mouthpiece, of the casing. Components in the cartridge are taken into the mouthpiece by air passing through the cartridge in the casing and then inhaled by users. Existing casings are generally disposable and made from plastic, thus being neither environmentally-friendly nor healthy; the air output cannot be controlled, and the quantity of air inhaled by users is difficult to control; and liquid in the cartridge is prone to volatilization and escape, leading to a short service life of the cartridge.

### BRIEF SUMMARY OF THE INVENTION

To solve the problems that existing casings are not environmentally-friendly, the quantity of air inhaled by users is difficult to control and the cartridge has a short service life due to volatilization and escape of liquid in the cartridge, the present invention provides a fresh air device. A specific technical solution is as follows:

A fresh air device includes a casing and a cartridge arranged in the casing, and further includes: an air inlet valve, arranged at one end of the casing; an air outlet valve detachably arranged at the other end of the casing; and a mouthpiece arranged on the air outlet valve.

Preferably, the air inlet valve includes: an air inlet valve seat, provided with a first adjusting threaded rod, an air inlet hole and an air inlet valve hole connected to the air inlet hole; an air inlet valve core, movably arranged in the air inlet valve hole and opposite to the air inlet hole; an air inlet base, provided with a first adjusting threaded hole connected to the first adjusting threaded rod and a first valve hole connected to the first adjusting threaded hole and the air inlet valve hole; and an air inlet spring, movably arranged in the first valve hole and connected to the air inlet valve core to allow the air inlet valve core to movably abut against a bottom of the air inlet hole.

Preferably, the air outlet valve includes: an air outlet valve seat, provided with a second adjusting threaded rod, a second valve hole and an air outlet valve hole connected to the second valve hole; an air outlet base, arranged on the casing and provided with a second adjusting threaded hole connected to the second adjusting threaded rod and a third valve hole connected to the second adjusting threaded hole; an air outlet valve core, movably arranged in the air outlet valve hole and opposite to the second valve hole; and an air outlet spring, movably arranged in the second valve hole and connected to the air outlet valve core to allow the air outlet valve core to movably abut against a bottom of the second valve hole.

Further, a slot allowing the cartridge to be inserted therein is formed in the air outlet valve seat, and the slot is connected to the air outlet valve hole.

Wherein, an air outlet duct connected to the air outlet valve hole and the second valve hole is formed in the air outlet valve seat.

Preferably, the fresh air device further includes an inner tube. The inner tube is arranged in the casing and has two ends respectively connected to the air outlet valve and the air inlet valve. The cartridge is arranged in the inner tube.

Preferably, the fresh air device further includes: a connecting base, arranged at one end of the casing; and a connecting magnet, arranged on the air outlet valve and opposite to the connecting base to attract the air outlet valve onto the connecting base by means of magnetic force.

Preferably, the fresh air device further includes steel balls. The steel balls are arranged on the air outlet valve, opposite to ball sockets in the connecting base and inserted into the ball sockets.

Preferably, the fresh air device further includes: a bearing, arranged on the mouthpiece; and a rotating base, arranged on the bearing and located between the mouthpiece and the air outlet valve.

A fresh air device assembly includes: a fresh air device; and a retaining base, provided with a retaining magnet used for attracting the fresh air device onto a top of the retaining base by means of magnetic force.

Compared with the prior art, the present invention has the following beneficial effects:

According to the fresh air device provided by the present invention, the air inlet valve and the air outlet valve are arranged at two ends to seal the casing and control the overall gas quantity, such that liquid in the cartridge may be effectively prevented from being volatilized and escaping, thus prolonging the service life of the cartridge; moreover, the overall gas quantity may be easily controlled as required, and the fresh air device is reusable and more environmentally friendly.

### BRIEF DESCRIPTION OF THE SEVERAL VIEWS OF THE DRAWINGS

FIG. 1 is a schematic structural diagram of a fresh air device;

FIG. 2 is a sectional view of the fresh air device;

FIG. 3 is a schematic structural diagram of an air inlet valve;

FIG. 4 is a schematic structural diagram of an air outlet valve;

FIG. 5 is a schematic structural diagram of an air outlet valve seat;

FIG. 6 is a schematic structural diagram of a casing and a connecting base;

FIG. 7 is a schematic assembly diagram of the air outlet valve and a mouthpiece;

FIG. 8 is a sectional view of FIG. 7;

FIG. 9 is a schematic structural diagram of a fresh air device assembly;

FIG. 10 is a sectional view of the fresh air device assembly.

### DETAILED DESCRIPTION OF THE INVENTION

The present invention is further described below in conjunction with accompanying drawings.

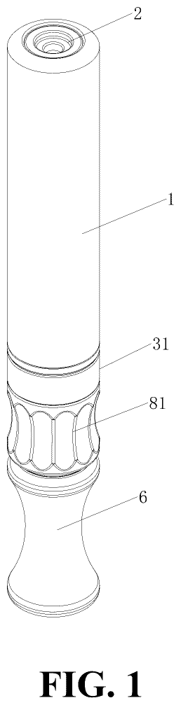

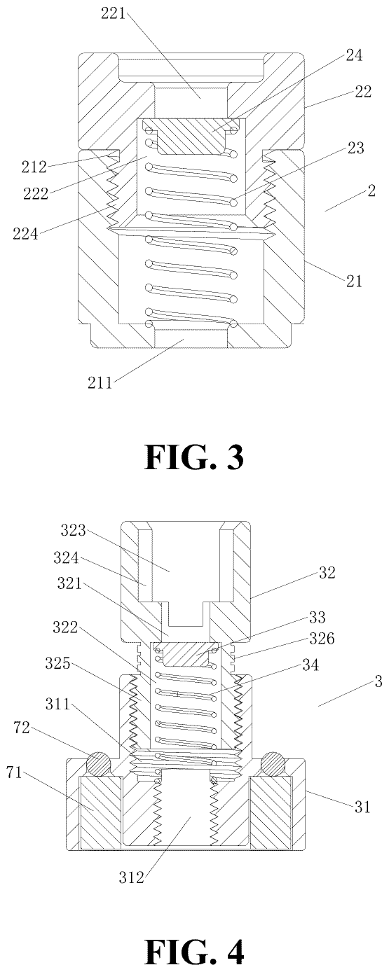

As shown in FIGS. 1-8, a fresh air device includes a casing 1, a cartridge 5, an air inlet valve 2, an air outlet valve 3 and a mouthpiece 6. The air inlet valve 2 and the air outlet valve 3 are respectively mounted at two ends of the casing 1. The air outlet valve 3 is detachably connected to the casing 1. The cartridge 5 is arranged in the casing 1 and located between the air outlet valve 3 and the air inlet valve 2. The mouthpiece 6 is mounted on the air outlet valve 3.

By means of the air inlet valve 2 and/or the air outlet valve 3, the air output is controlled, and the two ends of the casing 1 are sealed. In this way, the air output may be controlled, and liquid in the cartridge 5 may be prevented from being volatilized and escaping, thus prolonging the service life of the cartridge 5, that is, prolonging the use time of the cartridge 5.

When the air inlet valve 2 is adjusted, the air input may be controlled, which may in turn realize control of the air output at the rear end.

When the air outlet valve 3 is adjusted, the air output may be controlled.

Generally, only the air outlet valve 3 needs to be adjusted, and the air inlet valve 2 merely functions as a check valve to seal the air inlet end to prevent internal gas from escaping, thus prolonging the service life of the cartridge 5. The air inlet valve 2 and the air outlet valve 3 may be adjusted at the same time.

The air outlet valve 3 is detachably mounted at the bottom of the casing 1, such that the air outlet valve 3 may be pulled out to change the cartridge 5, thus making the fresh air device reusable, reducing the use cost and realizing environmental friendliness.

An air output scale 326 is arranged on a second adjusting threaded rod 325, such that the air output may be controlled easily.

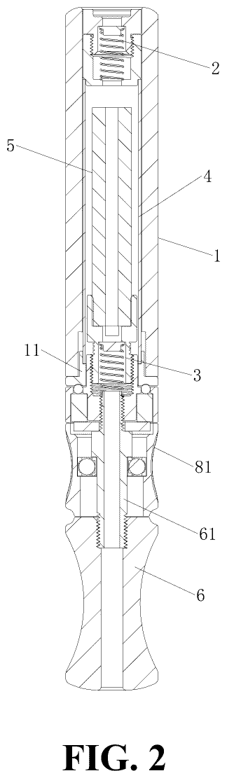

As shown in FIG. 2, the air inlet valve 2 includes an air inlet valve seat 22, an air inlet valve core 24, an air inlet base 21 and an air inlet spring 23. An air inlet hole 221 is formed in the top of the air inlet valve seat 22. A first adjusting threaded rod 224 is arranged at the bottom of the air inlet valve seat 22. An air inlet valve hole 222 is formed in the first adjusting threaded rod 224. The air inlet valve hole 222 is connected to the air inlet hole 221 and coaxial with the air inlet hole 221, and the diameter of the air inlet hole 221 is less than the diameter of the air inlet valve hole 222, such that a stepped hole is formed. The air inlet valve core 24 is movably inserted into the air inlet valve hole 222 and is arranged opposite to the air inlet hole 221. A first adjusting threaded hole 212 is formed in the top of the air inlet base 21. A first valve hole 211 is formed in the bottom of the first adjusting threaded hole 212. The diameter of the first valve hole 211 is less than the diameter of the first adjusting threaded hole 212, such that a stepped hole is formed. The first adjusting threaded rod 224 is mounted in the first adjusting threaded hole 212, such that the air inlet valve seat 22 and the air inlet base 21 are connected. The air inlet spring 23 is movably inserted into the first valve hole 211, and two ends of the air inlet spring 23 respectively abut against the air inlet valve core 24 and the bottom of the first adjusting threaded hole 212, such that the air inlet valve core 24 movably abuts against the bottom of the air inlet hole 221 to form a check valve structure. Air in the outside is only allowed to enter the casing 1, and gas in the casing 1 cannot flow out via the air inlet valve core 24, such that unidirectional sealing is realized. By rotating the first adjusting threaded rod 224, the amount of compression of the air inlet spring 23 may be adjusted to regulate the pressure in the air intake process so as to control the air input. The air inlet base 21 is fixed to and seals the top of the casing 1.

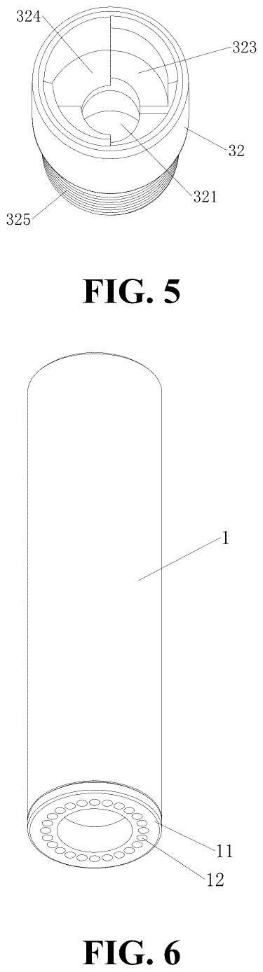

As shown in FIGS. 4 and 5, the air outlet valve 3 includes an air outlet valve seat 32, an air outlet base 31, an air outlet valve core 33 and an air outlet spring 34. A second valve hole 321 is formed in the top of the air outlet valve seat 32. The second adjusting threaded rod 325 and an air outlet valve hole 322 formed in the second adjusting threaded rod 325 are arranged at the bottom of the air outlet valve seat 32. The air outlet valve hole 322 is coaxial with the second valve hole 321 and connected to the second valve hole 321, and the diameter of the air outlet valve hole 322 is greater than the diameter of the second valve hole 321, such that a stepped hole is formed. A second adjusting threaded hole 311 is formed in the top of the air outlet base 31. A third valve hole 312 is formed in the bottom of the second adjusting threaded hole 311, and the diameter of the third valve hole 312 is less than the diameter of the second adjusting threaded hole 311, such that a stepped hole is formed. The air outlet valve seat 32 is mounted at the bottom of the casing 1, and the second adjusting threaded hole 311 is connected to the second adjusting threaded rod 325. The air outlet valve core 33 is movably inserted into the air outlet valve hole 322 and is arranged opposite to the second valve hole 321. The air outlet spring 34 is movably inserted into the second valve hole 321, and two ends of the air outlet spring 34 respectively abut against the air outlet valve core 33 and the bottom of the second adjusting threaded hole 311, such that the air outlet valve core 33 movably abuts against the bottom of the second valve hole 321 to form a check valve structure. In this way, gas in the casing 1 is only allowed to be discharged. When the fresh air device is not used, the air outlet valve hole 322 is sealed by means of the air outlet spring 34 to prevent gas in the casing 1 from escaping, such that continuous evaporation and escape of liquid on the cartridge 5 located in the casing 1 are avoided, thus prolonging the service life of the cartridge 5. By rotating the second adjusting threaded rod 325, the amount of compression of the air outlet spring 34 may be adjusted to control the air output.

To secure the cartridge 5, a slot 232 is formed in the top of the air outlet valve seat 32, the slot 232 is connected to and coaxial with the second valve hole 321, and the diameter of the slot 232 is greater than the diameter of the second valve hole 321. The cartridge 5 is inserted into the slot 232 to be secured.

To prevent the second valve hole 321 from being blocked by the cartridge 5, an air outlet duct 324 connected to the air outlet valve hole 322 and the second valve hole 321 is formed in the air outlet valve seat 32.

To improve the comfort in use and the aesthetics, the fresh air device further includes an inner tube 4. The inner tube 4 is mounted in the casing 1, two ends of the inner tube 4 are respectively connected to the air outlet valve 3 and the air inlet valve 2, and the cartridge 5 is arranged in the inner tube 4. The inner tube 4 may be made from stainless steel, and the cartridge 5 may be made from wood. The inner tube 4 made from stainless steel guarantees the sealing performance, facilitates assembly and disassembly of the air outlet valve 3 and the air inlet valve 2, and also facilitates cleaning. In this case, the casing 1 may be made from wood.

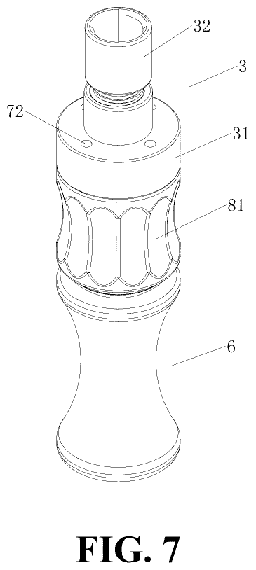

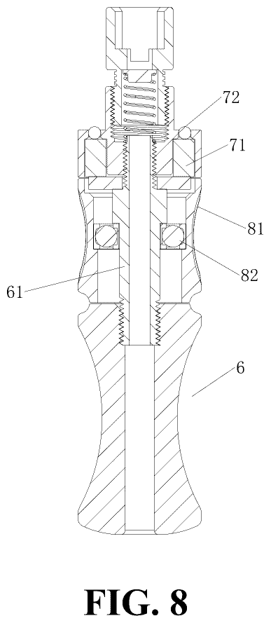

As shown in FIGS. 7 and 8, a hollow connecting shaft 61 is arranged on the mouthpiece 6, and two ends of the connecting shaft 61 are respectively connected to the mouthpiece 6 and the air outlet valve seat 32 by means of threads.

As shown in FIG. 2, to facilitate assembly and disassembly of the mouthpiece 6 and the air outlet valve 3, the fresh air device further includes a connecting base 11 and a connecting magnet 71. The connecting base 11 is fixed to the bottom of the casing 1, connected to the inner tube 4, and made from iron. The connecting magnet 71 is mounted on the air outlet valve seat 32, arranged opposite to the connecting base 11 and used for attracting the air outlet valve seat 32 onto the connecting base 11 by means of magnetic force.

To improve the interestingness and playability to relieve stress, the fresh air device further includes steel balls 72 fixed to the air outlet valve seat 32 in a ring array. A plurality of ball sockets 12 are formed in the connecting base 11 in a ring array, and the ball sockets 12 are opposite to the steel balls 72 to allow the steel balls 72 to be inserted therein. When the air outlet valve seat 32 is rotated, “tap-tap” sounds will be produced to remind users that the steel balls 72 are inserted into the ball sockets 12 in place, and stress may be relieved at the same time.

To further improve the playability to provide emotional value and relieve stress, the fresh air device further includes a bearing and a rotating base 81. The bearing is fixed to the connecting shaft 61 and connected to the rotating base 81. The rotating base 81 is located between the air outlet valve seat 32 and the mouthpiece 6. Users may hold the rotating base 81 to rotate the fresh air device.

Different cartridges 5 may be adopted to realize different functions, such as clearing away lung heat, nourishing the throat and refreshing breath, stress may be relieved, and many other functions such as smoking cessation, playing and appreciating, and wood rubbing may also be realized. The fresh air device is simple in overall structure, low in production cost, easy to use, environmentally friendly and reusable, and essential oil in the fresh air device is unlikely to leak.

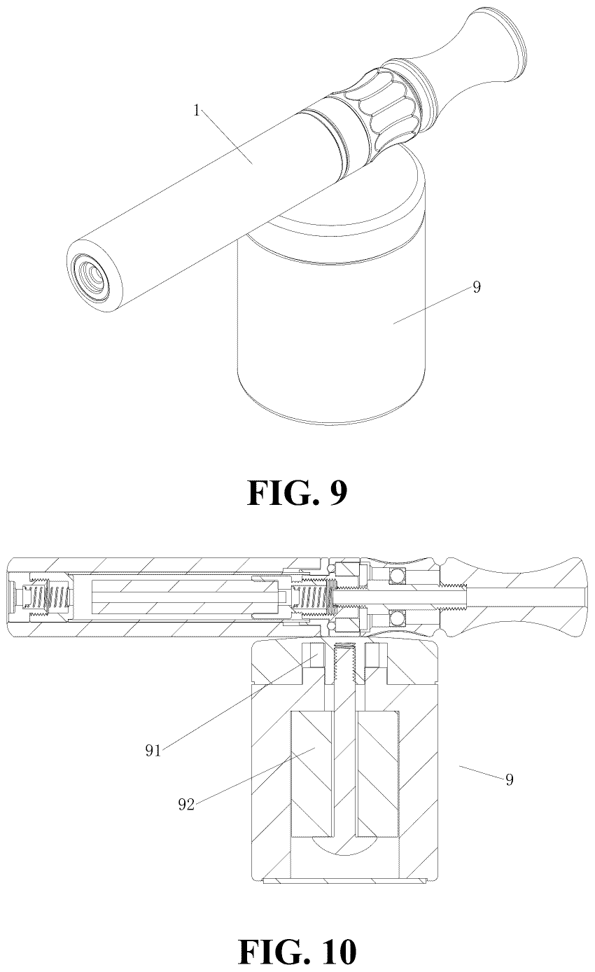

As shown in FIGS. 9 and 10, a fresh air device assembly includes a fresh air device and a retaining base 9. The retaining base 9 attracts the fresh air device by means of magnetic force, such that the fresh air device may be secured easily; and the fresh air device is suspended rather than being directly placed on a table, thus preventing a mouthpiece 6 from being polluted and preventing the fresh air device from rolling.

A retaining magnet 91 is arranged in the retaining base 9, and the retaining magnet 91 is able to attract the fresh air device.

To improve attractive force, the retaining magnet 91 is arranged opposite to a connecting magnet 71 in the fresh air device. The retaining magnet 91 is a ring magnet to enlarge the attraction area, such that the fresh air device may be easily attracted by the retaining magnet 91.

To prevent the fresh air device and the retaining base 9 from toppling, a weight 92 is arranged in the retaining base 9. The weight 92 increases the weight of the retaining base 9 to prevent the retaining base 9 moving synchronously when the fresh air device is picked up, such that the retaining base 9 may be stably retained on a table.

The technical principle of the present invention is described above in conjunction with specific embodiments. The above description is merely used to explain the principle of the present invention and should not be interpreted as limitations of the protection scope of the present invention in any way. All other specific embodiments that may be obtained by those skilled in the art based on the above explanation without creative labor should also fall within the protection scope of the claims of the present invention.

What is claimed is:

1. A fresh air device, comprising a casing (1) and a cartridge (5) arranged in the casing (1), wherein the fresh air device further comprises:
an air inlet valve (2), arranged at one end of the casing (1);
an air outlet valve (3), detachably arranged at the other end of the casing (1); and
a mouthpiece (6), arranged on the air outlet valve (3).

2. The fresh air device according to claim 1, wherein the air inlet valve (2) comprises:
an air inlet valve seat (22), provided with a first adjusting threaded rod (224), an air inlet hole (221) and an air inlet valve hole (222) connected to the air inlet hole (221);
an air inlet valve core (24), movably arranged in the air inlet valve hole (222) and opposite to the air inlet hole (221);
an air inlet base (21), provided with a first adjusting threaded hole (212) connected to the first adjusting threaded rod (224) and a first valve hole (211) connected to the first adjusting threaded hole (212) and the air inlet valve hole (222); and
an air inlet spring (23), movably arranged in the first valve hole (211) and connected to the air inlet valve core (24) to allow the air inlet valve core (24) to movably abut against a bottom of the air inlet hole (221).

3. The fresh air device according to claim 1, wherein the air outlet valve (3) comprises:
an air outlet valve seat (32), provided with a second adjusting threaded rod (325), a second valve hole (321) and an air outlet valve hole (322) connected to the second valve hole (321);
an air outlet base (31), arranged on the casing (1) and provided with a second adjusting threaded hole (311) connected to the second adjusting threaded rod (325) and a third valve hole (312) connected to the second adjusting threaded hole (311);
an air outlet valve core (33), movably arranged in the air outlet valve hole (322) and opposite to the second valve hole (321); and
an air outlet spring (34), movably arranged in the second valve hole (321) and connected to the air outlet valve core (33) to allow the air outlet valve core (33) to movably abut against a bottom of the second valve hole (321).

4. The fresh air device according to claim 3, wherein a slot (232) allowing the cartridge (5) to be inserted therein is formed in the air outlet valve seat (32), and the slot (232) is connected to the air outlet valve hole (322).

5. The fresh air device according to claim 4, wherein an air outlet duct (324) connected to the air outlet valve hole (322) and the second valve hole (321) is formed in the air outlet valve seat (32).

6. The fresh air device according to claim 1, further comprising an inner tube (4), wherein the inner tube (4) is arranged in the casing (1) and has two ends respectively connected to the air outlet valve (3) and the air inlet valve (2), and the cartridge (5) is arranged in the inner tube (4).

7. The fresh air device according to claim 1, further comprising:
a connecting base (11), arranged at one end of the casing (1); and
a connecting magnet (71), arranged on the air outlet valve (3) and opposite to the connecting base (11) to attract the air outlet valve (3) onto the connecting base (11) by means of magnetic force.

8. The fresh air device according to claim 7, further comprising steel balls (72), wherein the steel balls (72) are arranged on the air outlet valve (3), opposite to ball sockets (12) in the connecting base (11) and inserted into the ball sockets (12).

9. The fresh air device according to claim 1, further comprising:
a bearing (82), arranged on the mouthpiece (6); and
a rotating base (81), arranged on the bearing (82) and located between the mouthpiece (6) and the air outlet valve (3).

10. A fresh air device assembly, comprising:
the fresh air device according to claim 1; and
a retaining base (9), provided with a retaining magnet (91) used for attracting the fresh air device onto a top of the retaining base (9) by means of magnetic force.
